(19) United States
(12) Patent Application Publication
(10) Pub. No.: US 2023/0265118 A1
(43) Pub. Date: Aug. 24, 2023

(54) SALT OF CURCUMIN MONOGLUCURONIDE

(71) Applicant: THERABIOPHARMA INC., Kawasaki-shi (JP)

(72) Inventors: Atsushi IMAIZUMI, Kawasaki-shi (JP); Hitomi UMETA, Kawasaki-shi (JP); Yasumichi FUKUDA, Zama-shi (JP)

(73) Assignee: THERABIOPHARMA INC., Kawasaki-shi (JP)

(21) Appl. No.: 18/004,023

(22) PCT Filed: Jul. 2, 2021
(86) PCT No.: PCT/JP2021/025112

§ 371 Date: Dec. 30, 2022

(30) Foreign Application Priority Data
Jul. 2, 2020 (JP) 2020-115074

(51) Int. Cl. C07H 15/207

(52) U.S. Cl. CPC C07H 15/207

(57) ABSTRACT

Provided are a derivative of curcumin monoglucuronide excellent in solubility and chemical stability, which can be provided as pharmaceuticals, and a method for producing the same. A salt of curcumin monoglucuronide represented by Chemical Formula 1,

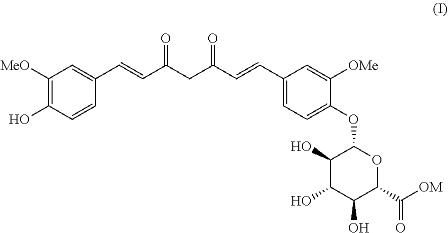

wherein M represents a pharmaceutically acceptable cation, a hydrate thereof, or a solvate thereof.

### FIELD OF THE INVENTION

The present invention relates to a salt of curcumin monoglucuronide and the like.

### BACKGROUND OF THE INVENTION

In recent years, it has been revealed that curcumin has a pharmacological action such as a tumorigenesis inhibitory action, an anti-oxidant action, an anti-inflammatory action, a cholesterol-lowering action, an anti-allergic action, a brain disease preventive action, and a heart disease preventive and therapeutic action, and a use of the curcumin in substances, for example, foods (for example, functional foods), pharmaceuticals, and cosmetics has been studied.

However, since the curcumin is hardly soluble in water, it is known that the curcumin is hardly absorbed into a body even in oral administration as it is. In addition, it is known that most of curcumin slightly absorbed by oral administration is present in blood as a curcumin conjugate conjugated with glucuronic acid and/or sulfuric acid, and free curcumin is only slightly present in the blood (Non Patent Literature 1).

Curcumin monoglucuronide is known as a glucuronic acid conjugate that is one of in vivo metabolites of the curcumin, and is used in metabolic studies of the curcumin and studies on development as the pharmaceuticals (Non Patent Literatures 2 to 5 and Patent Literature 1). For these purposes of use, production of the curcumin monoglucuronide via chemical methods has been reported (Non Patent Literatures 6 and 7, and Patent Literatures 1 and 2). The curcumin monoglucuronide, which is a free carboxylic acid form, has been required to be further improved in aspects such as solubility and chemical stability for development as pharmaceuticals.

### CITATION LIST

### Patent Literature

Patent Literature 1: WO 2018/003857 A
Patent Literature 2: JP 2015-054846 A

### Non Patent Literature

Non Patent Literature 1: Kei et al., Clinical Chemistry, 2009, 38:59-68
Non Patent Literature 2: FASEB Journal (2019), 33(8), 9453-9465
Non Patent Literature 3: Molecular Pharmaceutics (2019), 16(5), 1881-1889
Non Patent Literature 4: The Journal of Nutritional Biochemistry (2019), 63, 150-156
Non Patent Literature 5: Journal of Natural Products (2019), 82(3), 500-509
Non Patent Literature 6: Bioorganic & Medicinal Chemistry (2014), 22, 435-439.
Non Patent Literature 7: Antioxidants (2015), 4, 750-767.

### SUMMARY OF THE INVENTION

However, it has been found that the curcumin monoglucuronide has problems in terms of water solubility and storage stability in order to be used as pharmaceuticals.

An object of the present invention is to provide a derivative of the curcumin monoglucuronide excellent in solubility and chemical stability, which can be provided as pharmaceuticals, and a method for producing the same.

Therefore, as a result of intensive studies to solve the problems, it was surprisingly found by the present inventors that, as compared with the curcumin monoglucuronide which is the free carboxylic acid form, a salt of curcumin monoglucuronide has dramatic improvement in both solubility and chemical stability, and is excellent in applicability as pharmaceuticals, and accordingly, the present invention was completed.

The present invention provides the following [1] to [9].

[1] A salt of curcumin monoglucuronide represented by Chemical Formula (I) below,

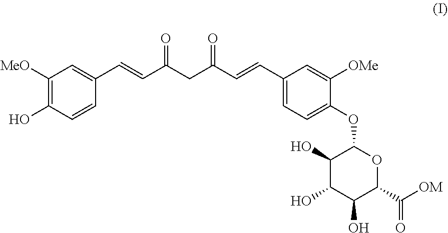

wherein M represents a pharmaceutically acceptable cation,

a hydrate thereof, or a solvate thereof.

[2] A compound according to [1], M being a sodium ion, a potassium ion, or an ammonium ion.

[3] A production method of the compound according to [1], wherein a curcumin monoglucuronide is reacted with a base represented by Chemical Formula (II) below,

Mm-Y (II)

wherein M represents a pharmaceutically acceptable cation, Y represents an anion, and m represents an integer from 1 to 3.

[4] The production method according to [3], wherein M is a sodium ion, a potassium ion or an ammonium ion, Y is a hydroxide ion, and m is 1.

[5] A pharmaceutical composition comprising the compound according to [1] or [2].

[6] The pharmaceutical composition according to [5], being a pharmaceutical composition for parenteral administration.

[7] The compound according to [1] or [2] for treating and/or preventing diseases.

[8] Use of the compound according to [1] or [2] for producing pharmaceuticals for treating and/or preventing diseases.

[9] A method for treating and/or preventing diseases, wherein the compound according to [1] or [2] is administered.

### Advantageous Effects of Invention

The salt of the curcumin monoglucuronide of the present invention exhibits excellent solubility and chemical stability, and thus is useful as pharmaceuticals, particularly pharmaceuticals via parenteral administration.

### DETAILED DESCRIPTION OF THE INVENTION

A salt of curcumin monoglucuronide of the present invention is represented by Chemical Formula (I) below,

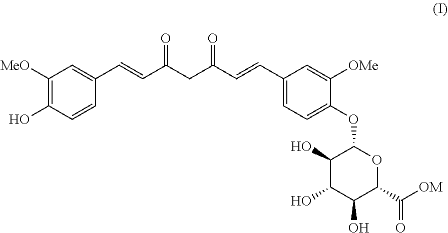

wherein M represents a pharmaceutically acceptable cation.

M is not particularly limited as long as it is a pharmaceutically acceptable cation, and examples thereof include an alkali metal ion such as a sodium ion and a potassium ion, an alkaline earth metal ion such as a magnesium ion and a calcium ion, and an ammonium ion. Among them, from a viewpoint of the solubility and the chemical stability of the compound of Chemical Formula (I), the sodium ion, the potassium ion, or the ammonium ion is more preferable, and the sodium ion is even more preferable.

A hydrate of the salt of the curcumin glucuronide is not particularly limited as long as a molecule of the salt of the curcumin glucuronide is hydrated with any number of water molecules. The hydrate of the salt of the curcumin glucuronide are preferably, for example, curcumin glucuronide sodium salt monohydrate or curcumin glucuronide sodium salt dihydrate.

A solvate of the salt of the curcumin glucuronide include, for example, an ethanol solvate.

In the salt of the curcumin monoglucuronide represented by Chemical Formula (I) above, tautomers represented by general Chemical Formulas (I-a) and (I-b) below may exist. However, the tautomers may reach an equilibrium state in which any tautomer coexists, and are not treated as a separate substance.

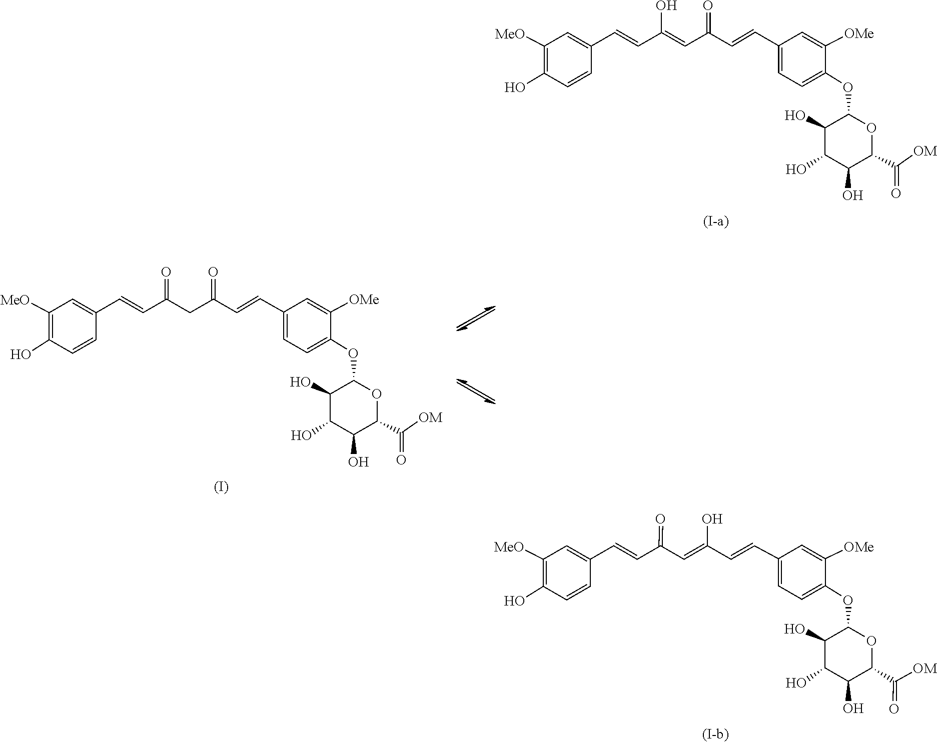

Examples of preferred compounds of the present invention include:

ammonium curcumin monoglucuronide [ammonium(2S,3S,4S,5R,6S)-3,4,5-trihydroxy-6-(4-((1E,6E)-7-(4-hydroxy-3-methoxyphenyl)-3,5-dioxohepta-1,6-dien-1-yl)-2-methoxyphenoxy)tetrahydro-2H-pyran-2-carboxylate],

potassium curcumin monoglucuronide [potassium(2S,3S,4S,5R,6S)-3,4,5-trihydroxy-6-(4-((1E,6E)-7-(4-hydroxy-3-methoxyphenyl)-3,5-dioxohepta-1,6-dien-1-yl)-2-methoxyphenoxy)tetrahydro-2H-pyran-2-carboxylate],

sodium curcumin monoglucuronide [sodium(2S,3S,4S,5R,6S)-3,4,5-trihydroxy-6-(4-((1E,6E)-7-(4-hydroxy-3-methoxyphenyl)-3,5-dioxohepta-1,6-dien-1-yl)-2-methoxyphenoxy)tetrahydro-2H-pyran-2-carboxylate], etc.

The salt of the curcumin monoglucuronide represented by Chemical Formula (I) can be produced by reacting free curcumin monoglucuronide with a base represented by Chemical Formula (II) below:

Mm-Y (II)

(wherein M represents a pharmaceutically acceptable cation, Y represents an anion, and m represents an integer from 1 to 3).

Free curcumin monoglucuronide can be obtained, for example, by the method described in Patent Literature 1.

Examples of the anion of Y in general Chemical Formula (II) include a hydroxide ion (OH⁻), a carbonate ion (CO₃²⁻), a bicarbonate ion (HCO₃⁻), a phosphate ion (PO₄³⁻), etc.

Among them, the anion of Y is preferably a hydroxide ion (OH⁻).

In general Chemical Formula (II), m represents an integer from 1 to 3, and can be appropriately determined according to M.

A combination of M, Y, and m are preferably NaOH, KOH, NH₄OH, Na₂CO₃, and NaHCO₃, more preferably NaOH, KOH, NH₄OH, and NaHCO₃, even more preferably NaOH and NaHCO₃.

A reaction above is preferably performed in the presence of a solvent, and such a solvent may contain water as desired, and examples thereof include acetone, acetonitrile, ethyl acetate, chloroform, N,N-dimethylformamide, ethanol, isopropanol, nitromethane, dimethyl carbonate, methanol, methyl tert-butyl ether, tetrahydrofuran, diisopropyl ether, cyclohexane, butanol, water, 3-pentanone, toluene, chlorobenzene, isobutyl acetate, etc.

The reaction can be performed with free curcumin monoglucuronide and the base of Chemical Formula (II) in the presence or absence of a solvent at a temperature from 0° C. to 100° C., preferably at a normal temperature (5° C. to 35° C.)

After completion of the reaction above, an obtained salt can be isolated from a reaction mixture by any conventional means such as precipitation, concentration, or centrifugation.

Since the salt of the curcumin monoglucuronide of the present invention, the hydrate thereof, and the solvate thereof thus obtained have high water solubility and favorable chemical stability, they are useful as the pharmaceuticals for preventing or treating diseases that can be prevented or treated by administration of the curcumin.

Accordingly, the salt of the curcumin monoglucuronide of the present invention can be a pharmaceutical composition containing the salt. In addition, the salt of the curcumin monoglucuronide can be a compound for treating and/or preventing various diseases. In addition, the salt of the curcumin monoglucuronide is also useful for producing the pharmaceuticals for treating and/or preventing various diseases. In addition, the method for treating and/or preventing various diseases characterized by administering the salt of the curcumin monoglucuronide can also be provided.

As described above, the curcumin has a pharmacological action such as a tumorigenesis inhibitory action, an anti-oxidant action, an anti-inflammatory action, a cholesterol-lowering action, an anti-allergic action, a brain disease preventive action, and a heart disease preventive and therapeutic action. Accordingly, the salt of the curcumin glucuronide of the present invention is useful as pharmaceuticals such as anti-cancer pharmaceuticals, anti-inflammatory pharmaceuticals, cholesterol-lowering pharmaceuticals, anti-allergic pharmaceuticals, cognitive function improving pharmaceuticals, heart disease preventive and therapeutic pharmaceuticals.

An administration route of the pharmaceutical composition containing the salt of the curcumin glucuronide of the present invention is not particularly limited, and may be an oral administration or a parenteral administration. However, since the salt of the curcumin glucuronide of the present invention has high water solubility and favorable chemical stability, it is possible to maintain blood concentration of free curcumin at a high value for a long time by the parenteral administration similar to the curcumin glucuronide, to effectively obtain the pharmacological actions of the curcumin. Accordingly, the pharmaceutical composition of the present invention is particularly useful as a pharmaceutical composition for parenteral administration.

A form of the pharmaceutical composition for parenteral administration containing the salt of the curcumin monoglucuronide of the present invention is not limited as long as it is administered parenterally. However, a composition for injection (an injection preparation) is particularly preferable. Examples of the composition for injection include a composition for intravenous administration and a composition for subcutaneous administration, and the composition for intravenous administration is more preferable.

For example, a content of the salt of the curcumin monoglucuronide in the pharmaceutical composition for parenteral administration is not particularly limited, and is preferably from 1 to 100 mass %, more preferably from 5 to 100 mass %, and even more preferably from 10 to 100 mass %.

In such a composition for parenteral administration, in addition to the salt of the curcumin monoglucuronide, substances like water, physiological saline, a pH adjusting agent, a saccharide, an acid, an alkali, a buffer, a tonicity agent, a stabilizer, a soothing agent, an antiseptic agent, and the like can be blended.

Examples of the saccharide include monosaccharides, disaccharides, trisaccharides, polysaccharides, sugar alcohols, etc. Examples of the acid and the alkali include a water-soluble inorganic acid, a salt of water-soluble inorganic acid, a water-soluble organic acid, a salt of water-soluble organic acid, an amino acid, a salt of amino acid, etc. In addition, the form of the composition for parenteral administration of the present invention may be in the form of a powder filler dissolved when use (a crystal or a lyophilized product), or in the form of an aqueous solution.

### EXAMPLES

Hereinafter, the present invention will be described more specifically with reference to examples. However, the scope of the present invention is not limited to specific aspects to be described below.

### Example 1 (Synthesis of a Sodium Salt from Free Curcumin Glucuronide)

A curcumin monoglucuronic acid conjugate (1.00 g) synthesized according to a known method (Patent Literature 1) was dissolved in methanol (20 mL), and 0.5 M sodium bicarbonate aqueous solution (4.0 mL) was added thereto. A precipitated solid was collected by filtration to obtain a sodium salt of the curcumin monoglucuronic acid conjugate (0.64 g, purity 99% or more).

### Example 2 (Stability)

A curcumin monoglucuronic acid conjugate (3.62 mg) and a sodium salt of the curcumin monoglucuronic acid conjugate (3.44 mg) were each dissolved in 15 mL of water-methanol (1:1). The prepared solution was left at room temperature for 35 days, and a residual ratio was determined by HPLC.

(1) Residual Ratio

The curcumin monoglucuronic acid conjugate: 80.8%

The sodium salt of the curcumin monoglucuronic acid conjugate: 93.8%

(2) Half-Life

The curcumin monoglucuronic acid conjugate: 126.1 days

The sodium salt of the curcumin monoglucuronic acid conjugate: 378.4 days

### Example 3 (Solubility)

When 100 mg of each of a curcumin monoglucuronic acid conjugate and a sodium salt of the curcumin monoglucuronic acid conjugate was dissolved in 1 mL of distilled water, the curcumin monoglucuronic acid conjugate was hardly dissolved, and 100 mg of the sodium salt of the curcumin monoglucuronic acid conjugate was dissolved in 1 mL of the distilled water. As a result, it was confirmed that the sodium salt of curcumin monoglucuronide had a requisite amount of solubility for injections.

1: A salt of curcumin monoglucuronide represented by Chemical Formula 1 below, 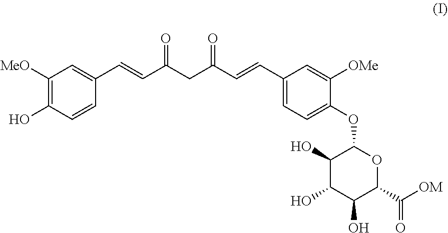
wherein M represents a pharmaceutically acceptable cation,
a hydrate thereof, or a solvate thereof.

2: A compound according to claim 1, M being a sodium ion, a potassium ion, or an ammonium ion.

3: A method of preparing the compound according to claim 1, wherein a curcumin monoglucuronide is reacted with a base represented by Chemical Formula (II) below,
Mm-Y (II)
wherein M represents a pharmaceutically acceptable cation, Y represents an anion, and in represents an integer from 1 to 3.

4: The method according to claim 3, wherein M is a sodium ion, a potassium ion or an ammonium ion, Y is a hydroxide ion, and m is 1.

5: A pharmaceutical composition comprising the compound according to claim 1.

6: The pharmaceutical composition comprising a solution of the compound according to claim 5, for parenteral administration.

7-9. (canceled)

10: A method of treating or preventing a disease with curcumin comprising administering the composition of claim 5

11: The method of claim 10 wherein the pharmaceutical composition comprises an aqueous solution.

12: The method of claim 11 wherein the composition is administered intravenously.
